(19) United States
(12) Patent Application Publication
(10) Pub. No.: US 2010/0234712 A1
(43) Pub. Date: Sep. 16, 2010

(54) DEVICE AND METHOD FOR ACCURATELY MEASURING CONCENTRATION OF BLOOD COMPONENT

(75) Inventors: Junichi SUGENOYA, Nagoya-shi (JP); Yuzo NAKASE, Moriya-shi (JP); Satoshi NAKAJIMA, Kyoto-shi (JP); Muneo TOKITA, Nagaokakyo-shi (JP)

Correspondence Address: Omron Healthcare Co. Ltd.;c/o Keating & Bennett, LLP, 1800 Alexander Bell Drive, Suite 200, Reston, VA, 20191, US

(73) Assignee: OMRON HEALTHCARE Co., Ltd., Kyoto-shi (JP)

(21) Appl. No.: 12/775,648

(22) Filed: May 7, 2010

Related U.S. Application Data
(63) Continuation of application No. PCT/JP2008/070036, filed on Nov. 4, 2008.

(30) Foreign Application Priority Data
Nov. 9, 2007 (JP) 2007-292407

(51) Int. Cl. A61B 5/145

(52) U.S. Cl. 600/365

(57) ABSTRACT

A measurement computation device arranged in a measurement device detects a first component and a second component from perspiration in a first component detection unit and a second component detection unit, and the respective concentration in the perspiration is calculated in a concentration calculation unit. In a conversion computation unit, the concentration of the first component in the perspiration is corrected using the concentration of the second component, and then converted to the concentration of the first component in the blood.

### BACKGROUND OF THE INVENTION

1. Field of the Invention

The present invention relates to devices and methods for measuring the concentration of a blood component, in particular, to a device and a method for measuring the concentration of a blood component using perspiration.

2. Description of the Related Art

A method of measuring the concentration of a component in blood such as blood glucose without collecting blood includes a method of measuring based on the concentration of a component contained in perspiration. For instance, U.S. Pat. No. 5,036,861 and Japanese Laid-Open Patent Publication No. 62-72321 describe such a method and device.

Specifically, as a method of forcibly perspiring, U.S. Pat. No. 5,036,861 discloses a medical agent introducing method, that is, a method of introducing the medical agent to a target area, and Japanese Laid-Open Patent Publication No. 62-72321 discloses a warming method, that is, a method of warming the target area. Japanese Laid-Open Patent Publication No. 62-72321 also describes that the perspiration sugar and the blood glucose are correlated.

However, a change in concentration of the sugar concentration in the perspiration is not necessarily correlated with the change in concentration of the blood glucose value. This is also apparent from the graph showing the correlation of the perspiration sugar and the blood glucose shown in Japanese Laid-Open Patent Publication No. 62-72321. The inventors found that a correlation is not established at the beginning of forced perspiration in particular. Furthermore, the correlation between perspiration sugar and blood glucose is not always constant, and sometimes changes when a certain period of time elapses, such as several days. Therefore, if the glucose concentration in the blood is estimated using the glucose concentration in the perspiration, a problem in that an accurate glucose concentration in the blood may not always be obtained arises. Similar problems arise when the blood component is a component other than sugar.

### SUMMARY OF THE INVENTION

In view of such problems, preferred embodiments of the present invention provide a device and a method capable of accurately measuring the concentration of a blood component using perspiration.

In accordance with a preferred embodiment of the present invention, a blood component concentration measurement device includes: a perspiration accelerating unit arranged to accelerate perspiration from a body surface or a measurement site; a first measurement unit arranged to measure concentration in the perspiration of a first component contained in the perspiration from the measurement site; a second measurement unit arranged to measure concentration in the perspiration of a second component, different from the first component, contained in the perspiration from the measurement site; a correction unit arranged to correct the concentration in the perspiration of the first component using the concentration in the perspiration of the second component; and a conversion unit arranged to convert the result corrected by the correction unit to the concentration of the first component in a blood of the body.

In accordance with another preferred embodiment of the present invention, a measurement method performed by a blood component concentration measurement device which includes an acquiring device arranged to acquire perspiration from a measurement site, a detection device arranged to detect a component in the perspiration, and a computation device arranged to perform computation using a value obtained from the component in the perspiration, the method including the steps of: detecting a first component from the perspiration with the detection device; calculating a concentration in the perspiration of the first component with the computation device; detecting a second component, different from the first component, from the perspiration with the detection device; calculating a concentration in the perspiration of the second component with the computation device; correcting the concentration in the perspiration of the first component using the concentration in the perspiration of the second component with the computation device; converting the result corrected in the correcting step to the concentration of the first component in the blood of the body with the computation device; and executing a process of outputting the concentration in the blood of the first component with the computation device.

According to various preferred embodiments of the present invention, the concentration of the blood component can be accurately measured using perspiration.

Other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a view showing a specific example of an outer appearance of a measurement device according to a preferred embodiment of the present invention, where (A) portion is a view showing a specific example of an outer appearance of a perspiration device and (B) portion is a measurement computation device 30.

FIG. 2A is a view showing a specific example of a mechanical configuration of the perspiration device according to a preferred embodiment of the present invention seen from the front surface.

FIG. 2B is a view showing a cross-section at a position of an arrow A of FIG. 2A of a mechanical configuration of the perspiration device according to a preferred embodiment of the present invention.

FIG. 3A is a view showing a specific example of a mechanical configuration of the measurement computation device according to a preferred embodiment of the present invention seen from the front surface.

FIG. 3B is a view showing a cross-section at a position of an arrow B of FIG. 3A of a mechanical configuration of the measurement computation device according to a preferred embodiment of the present invention.

FIG. 4 is a view describing one example of a method of conveying the perspiration from the perspiration collection region to the discarding liquid storage unit in the measurement computation device.

FIG. 5 is a view showing another specific example of a mechanical configuration of the measurement computation device.

FIG. 6 is a block diagram showing a specific example of the function configuration of the perspiration device according to a preferred embodiment of the present invention.

FIG. 7 is a block diagram showing a specific example of the function configuration of the measurement computation device according to a preferred embodiment of the present invention.

FIG. 8 is a view showing the change over time of the sugar concentration in the blood and the sugar concentration in the perspiration after the perspiration occurs.

FIG. 9 is a view showing the changes over time of the glutamic acid concentration in the perspiration after the perspiration occurs.

FIG. 10 is a flowchart showing the flow of the perspiring operation in the perspiration device according to a preferred embodiment of the present invention.

FIG. 11 is a flowchart showing a flow of the measurement computation operation in the measurement computation device according to a preferred embodiment of the present invention.

FIG. 12 is a view showing the changes over time of the concentration in the perspiration after the correction and the blood glucose value.

### DETAILED DESCRIPTION OF THE PREFERRED EMBODIMENTS

Hereafter, the preferred embodiments of the present invention will be described with reference to the drawings. In the following description, the same reference numerals are denoted for the same components and the configuring elements. The names and functions thereof are the same.

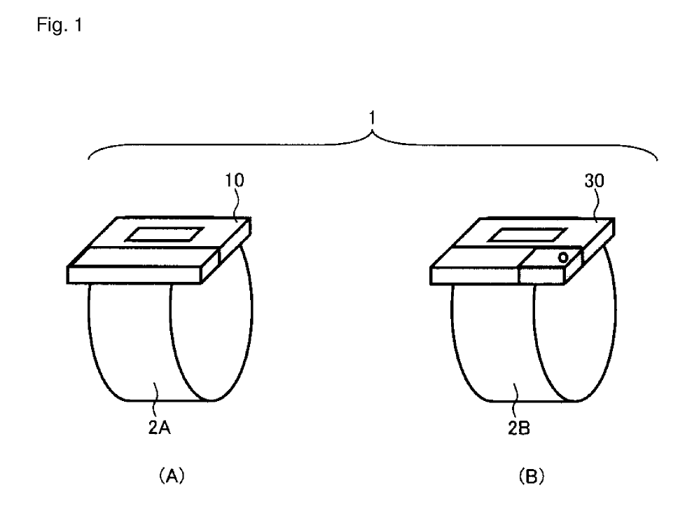

FIG. 1 is a view showing a specific example of an outer appearance of a blood component concentration measurement device (hereinafter abbreviated as measurement device) 1 according to the present preferred embodiment. The measurement device 1 includes a perspiration device 10 ((A) portion of FIG. 1) and a measurement computation device 30 ((B) portion of FIG. 1). The perspiration device 10 and the measurement computation device 30 are used by being attached to measurement sites such as a wrist and an ankle with belts 2A and, 2B, respectively.

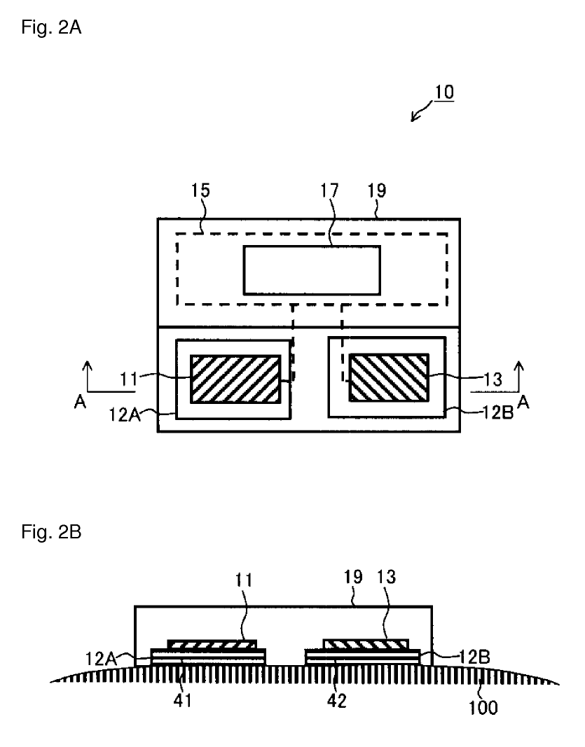

Specifically, with reference to FIG. 2A, the perspiration device 10 includes an introducing electrode 11 serving as an anode, and a reference electrode 13 serving as a cathode, inside a housing 19. The introducing electrode 11 and the reference electrode 13 are connected to a control circuit 15. A display 17 is arranged at a position that can be visually recognized in a state of being attached to the measurement site using the belt 2A on the housing 19 such as the surface shown on the upper side at the (A) portion of FIG. 1. The display 17 is also connected to the control circuit 15. FIG. 2A is a schematic view of the perspiration device 10 seen from the surface shown on the upper side at the (A) portion of FIG. 1. The surface shown in FIG. 2A is a front surface of the housing 19 of the perspiration device 10. An operation unit such as a button (not shown) is arranged at the front surface of the housing 19. The operation unit is also connected to the control circuit 15.

FIG. 2B is a schematic view of a mechanical configuration of the cross-section of the perspiration device 10 at the position shown with an arrow A in FIG. 2A. With reference to FIG. 2B, the introducing electrode 11 and the reference electrode 13 are arranged at positions close to the surface on the far side from the front surface of the housing 19 in the housing 19, that is, at the positions close to the skin 100 serving as the measurement site in a state where the perspiration device 10 is attached to the measurement site using the belt 2A. Medical agent regions 12A, 12B are arranged between the introducing electrode 11 and the skin 100 and between the reference electrode 13 and the skin 100, respectively, of the housing 19. The medical agent region 12A is set preferably with a member or material such that the perspiration accelerator contacts the skin, such as a sponge 41 including a liquid containing medical agent (perspiration accelerator) that accelerates perspiration, such as a pilocarpine solution, for example. The medical agent region 12B is preferably set with a buffer such as a sponge 42 containing buffer solution. The medical agent regions 12A, 12B may have a configuration in which the medical agent is injected as is, a configuration in which the gelatinized medical agent is set, or a configuration in which the medical agent absorbed into absorbent cotton and the like is set. The configurations of the medical agent regions 12A, 12B may be any configuration as long as the medical agent set in the medical agent regions 12A, 12B contact the skin 100 in a state where the perspiration device 10 is attached to the measurement site.

The control circuit 15 stores current values in advance. When a control signal for starting the perspiration is input from the operation unit, the control circuit 15 generates a DC current with a specified current value from the introducing electrode 11 to the reference electrode 13 according to the control signal.

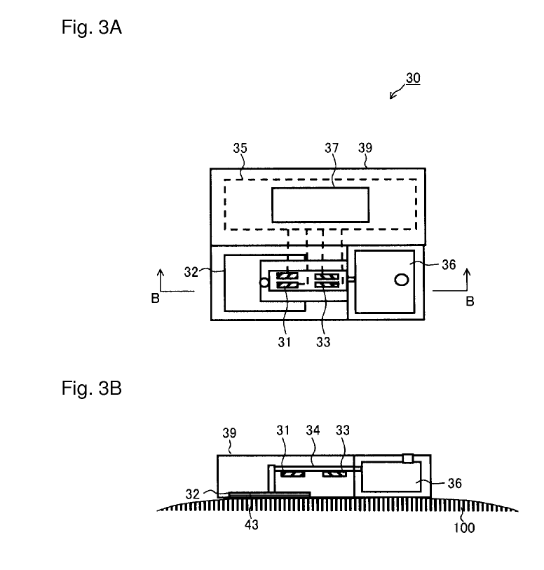

With reference to FIG. 3A, a measurement computation device 30 includes a first component detector 31 arranged to detect a first component in the perspiration and a second component detector 33 arranged to detect a second component inside a housing 39. The first component detector 31 and the second component detector 33 are connected to a control circuit 35. A display 37 is arranged at a position that can be visually recognized in a state of being attached to the measurement site using the belt 2B on the housing 39 such as the surface shown on the upper side at the (B) portion of FIG. 1. The display 37 is also connected to the control circuit 35. FIG. 3A is a schematic view of the measurement computation device 30 seen from the surface shown on the upper side at the (B) portion of FIG. 1. The surface shown in FIG. 3A is a front surface of the housing 39 of the measurement computation device 30. An operation unit such as a button (not shown) is arranged at the front surface of the housing 39. The operation unit is also connected to the control circuit 35.

FIG. 3B is a schematic view of a mechanical configuration of the cross-section of the measurement computation device 30 at the position shown with an arrow B in FIG. 3A. With reference to FIG. 3B, a perspiration collection region 32 is arranged at a position close to the surface on the far side from the front surface of the housing 39 in the housing 39, that is, at a position close to the skin 100 serving as the measurement site in a state where the measurement computation device 30 is attached to the measurement site using the belt 2B. The perspiration collection region 32 is preferably set with a member or material that is operative to collect perspiration from the skin 100, such as a sponge 43 for collecting perspiration. The perspiration collection region 32 may have a configuration of collecting perspiration directly from the skin 100, or a configuration in which a medical agent for gelatinizing the perspiration is set. The configuration of the perspiration collection region 32 may be any configuration as long as the perspiration can be collected from the skin 100 in a state where the measurement computation device 30 is attached to the measurement site. Furthermore, a discarding liquid storage unit 36 for storing discarded liquid after component detection is arranged inside the housing 39 of the measurement computation device 30. A conveyance path 34 is arranged to convey the perspiration from the perspiration collection region 32 to the discarding liquid storage unit 36 through the first component detector 31.

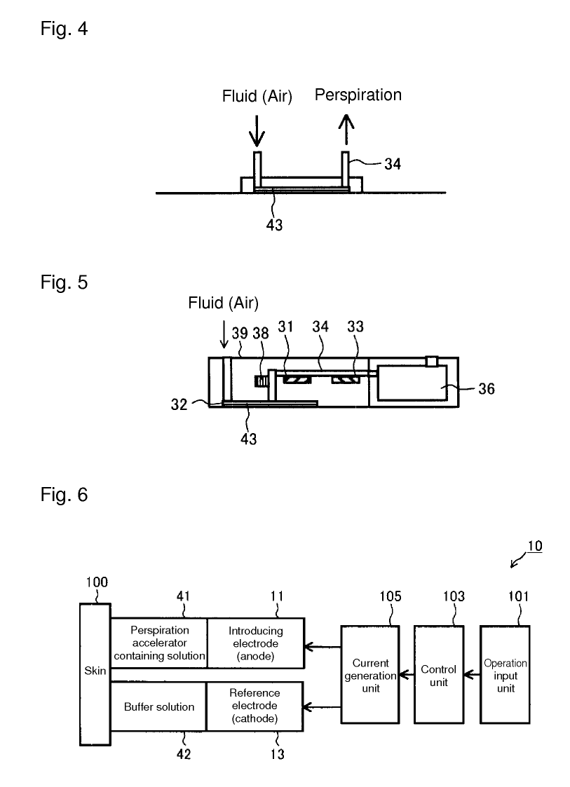

The present invention is not limited to the conveyance path 34 to convey the perspiration as described above, and a method of injecting fluid such as air from one side of the conveyance path 34 including the perspiration collection region 32 and pushing out the internal perspiration to the other side, as shown in FIG. 4, may be adopted.

The mechanical configuration shown in FIGS. 2A, 2B, 3A, 3B is a specific example, and the configurations of the perspiration device 10 and the measurement computation device 30 are not limited to the illustrated configurations. For instance, as another specific example of the configuration of the measurement computation device 30, a liquid sensor 38 arranged to detect the perspiration amount that is collected by the perspiration collection region 32 and that reached the conveyance path 34 may be provided, as shown in FIG. 5, to convey the perspiration in the conveyance path 34. In this case, when detecting that the collected perspiration amount reached a predetermined amount based on the detection signal from the liquid sensor 28, the control circuit 35 outputs a control signal to a mechanism for injecting fluid such as compressed air (not shown) to the conveyance path 34, and conveys the perspiration of the perspiration collection region 32 to the first component detector and the second component detector 33. Furthermore, the perspiration at the first component detector 31 and the second component detector 33 is conveyed to the discarding liquid storage unit 36 after component detection is performed in the first component detector 31 and the second component detector 33.

As another specific example of the configuration of the measurement device 1, the perspiration device 10 and the measurement computation device 30, which are separate devices, shown in FIG. 1 may be used by being combined and mounted on one belt 2. In this case, the control circuit and the display may be commonly used by the perspiration device 10 and the measurement computation device 30. With such a configuration, the perspiration device 10 and the measurement computation device 30 are attached to the same measurement site, and thus the perspiration can be efficiently collected from the same position as the portion where perspiration is accelerated by the perspiration device 30. As another configuration, in the display 17, the elapsed time from when the perspiring operation is started may be displayed when the perspiring operation starts in the perspiration device 10.

The first component is a component that becomes a target of calculating the blood concentration, and corresponds to a component in which the change in concentration in the perspiration and the change in concentration in the blood are related. Specifically, this corresponds to sugar (glucose), where the first component is sugar in the present preferred embodiment.

The second component is a component in the perspiration other than the first component. The second component is preferably a component in which relevance does not exist or the relevance is lower than a predetermined correlation coefficient between the change in concentration in the perspiration and the change in concentration in the blood. More preferably, the second component is a substance in which the rate of being reabsorbed by the perspiration tube until reaching the skin from the perspiratory gland is smaller than a predetermined value. A substance in which the rate of being reabsorbed by the perspiration tube is large includes water and sodium, and the second component is preferably not such components. Specifically, the second component satisfying such conditions includes, in addition to glutamic acid, other amino acids such as lysine, glutamine, and asparagine acid, calcium, and kalium if the first component is sugar, where the second component is glutamic acid in the present preferred embodiment.

The first component detector 31 and the second component detector 33 of the measurement computation device 30 have a configuration of detecting the component in the perspiration, but is not limited to a specific configuration. For instance, the component may be detected by measuring the wavelength of the radiation light, or an enzyme electrode method may be used. The configuration corresponding to the first component and the second component to be measured may be adopted. If the first component detector 31 and the second component detector 33 use the enzyme electrode method, the measurement computation device 30 can be miniaturized compared to other configurations such as the configuration of measuring the wavelength of the radiation light. The first component detector 31 in the present preferred embodiment may have a configuration combining glucose oxidase and electrode using the enzyme electrode method to detect sugar as the first component. The second component detector 33 preferably has a configuration in which L-glutamic acid glutamate oxidase and an electrode are combined using an enzyme electrode method to detect the glutamic acid as the second component.

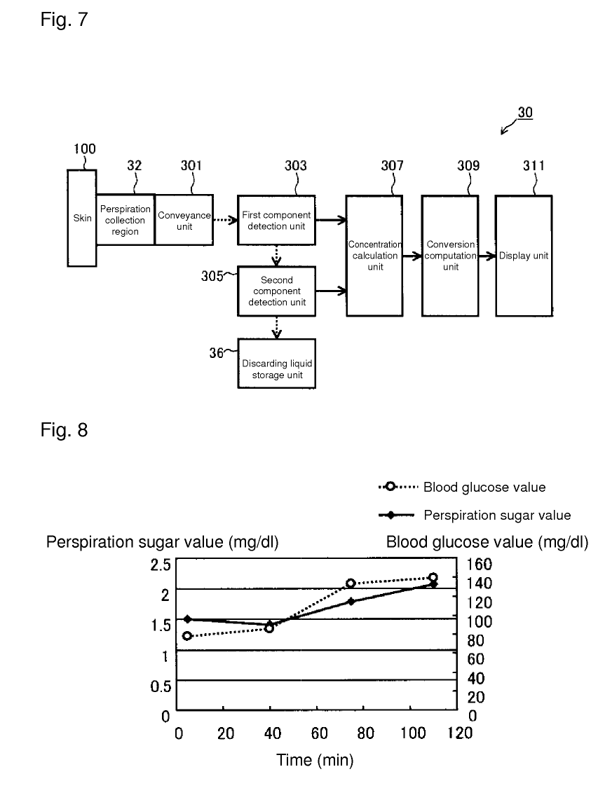

FIG. 6 and FIG. 7 are block diagrams showing a specific example of the function configuration for collecting the perspiration from the skin 100 and calculating the concentration of the first component in the blood using the concentrations of the first component and the second component in the perspiration in the measurement device 1 including the perspiration device 10 and the measurement computation device 30. FIG. 6 shows a specific example of the perspiration device 10. FIG. 7 shows a specific example of the function configuration of the measurement computation device 30. Each function shown in FIG. 6 and FIG. 7 is a function implemented when the control circuit 15 of the perspiration device 10 and the control circuit 35 of the measurement computation device 30 execute a predetermined control program. At least one portion of such functions maybe implemented by the mechanical configuration shown in FIGS. 2A, 2B or FIGS. 3A, 3B. The solid line arrow in FIG. 6 and FIG. 7 shows a flow of electric signal. The dotted line arrow in FIG. 7 shows the conveyance of perspiration.

With reference to FIG. 6, the function of the perspiration device 10 includes an operation input unit 101 for accepting the input of the operation signal from the operation unit (not shown in FIG. 1, and FIGS. 2A, 2B), a control unit 103, and a current generation unit 105.

The control unit 103 is mainly configured by the control circuit 15, and starts the perspiring operation based on the operation signal input from the operation input unit 101. The perspiring operation starts when the control unit 103 inputs the control signal for generating a current of a defined value based on the operation signal to the current generation unit 105. The current generation unit 105 is also mainly configured by the control circuit 15, and performs the process of generating the current of the defined value between the introducing electrode 11 and the reference electrode 13 according to the control signal. Through such process, the DC current flows from the introducing electrode 11 towards the reference electrode 13, passing through the skin 100 through the sponge 41 containing the pilocarpine solution or the solution containing the perspiration accelerator. Thus, the pilocarpine solution or the substance of the introducing electrode 11 is introduced by being infiltrated to under the skin, and acts on the perspiratory gland. Such a method of introducing the substance is referred to as an iontophoresis method.

When a predetermined time elapses from the start of the perspiring operation, the perspiration occurs from the perspiratory gland near the introducing electrode 11. When the pilocarpine solution is infiltrated after elapse of a constant time from the start of the perspiring operation in the perspiration device 10, the control signal 103 outputs a control signal to stop the generation of current to the current generation unit 105 according to the operation signal so as to terminate the perspiring operation from the operation input unit 101, and terminates the perspiring operation. The perspiring operation may be terminated when the control unit 103 detects elapse of a constant time from the start of the perspiring operation and outputs the control signal to stop the generation of current to the current generation unit 105.

With reference to FIG. 7, the function of the measurement computation device 30 includes a conveyance unit 301 arranged to convey the perspiration accommodated in the perspiration collection region 32, a first component detection unit 303 arranged to detect the first component in the perspiration, a second component detection unit 305 arranged to detect the second component, a concentration calculation unit 307 arranged to calculate the concentration of the first component and the second component in the perspiration based on the detection signal from the first component detection unit 303 and the second component detection unit 305, a conversion computation unit 309 arranged to perform a computation for obtaining the concentration of the first component in the blood using the calculation result, and a display unit 311 arranged to perform a process of displaying the computation result.

The conveyance unit 301 is configured by the conveyance mechanism as described above, and conveys the perspiration accommodated in the perspiration collection region 32 to the discarding liquid storage unit 36 through the first component detector and the second component detector 33. In the case of the configuration in which the measurement computation device 30 injects fluid such as compressed air to the conveyance path 34 to convey the perspiration accommodated in the perspiration collection region 32, the conveyance unit 301 includes a mechanism for injecting fluid to the conveyance path 34. Specifically, when injecting fluid by operation a mechanical configuration such as a pump, the conveyance unit 301 includes the mechanical configuration and the configuration of outputting a control signal for operating the configuration.

The first component detection unit 303 mainly includes the first component detector 31. The second component detection unit 305 mainly includes the second component detector 33. Such functions detect the first component or the second component using the first component detector 31 or the second component detector 33 from the perspiration conveyed by a conveyance unit 301, and input a detection signal corresponding to the detection amount to a concentration calculation unit 307.

The concentration calculation unit 307 is mainly configured by the control circuit 35, and calculates the concentration of the first component in the perspiration based on the detection signal input from the first component detection unit 303 according to a predetermined computation program. Similarly, the concentration calculation unit 37 calculates the concentration of the second component in the perspiration based on the detection signal input from the second component detection unit 305 according to a predetermined computation program. The signal indicating the calculated concentration is input to the conversion computation unit 309.

A conversion computation unit 309 is mainly configured from the control circuit 35, and performs a computation for converting the concentration of the first component in the perspiration to the concentration of the first component in the blood using the concentration of the second component according to a predetermined computation program. The computation result is input to the display unit 311, and a process of displaying the concentration of the first component in the blood on the display 37 as a computation result is performed at the display 37.

The principle of computation in the conversion computation unit 309 will now be described.

The behaviors of the component concentration in the blood and the component concentration in the perspiration are known to be substantially proportional. FIG. 8 is a view showing time change between the sugar concentration in the blood and the sugar concentration in the perspiration after perspiration. The change in concentration shown in FIG. 8 shows time change between the sugar concentration in the blood (blood glucose value) and the sugar concentration in the perspiration (perspiration sugar value) for every 40 minutes when sugar load is performed at a time point of 40 minutes from an empty stomach state and the blood glucose value is changed. As shown in FIG. 8, the blood glucose value and the perspiration sugar value are substantially proportional, assuming the first component is the sugar. However, as shown in FIG. 8, the perspiration sugar value behaves at high concentration with respect to the behavior of the blood glucose value at the beginning of perspiration such as until elapse of 40 minutes from perspiration in FIG. 8. Thereafter, the perspiration sugar value also rises in cooperation with rise in blood glucose value. The sugar concentration in the perspiration is also influenced by the state of skin of the relevant day (state of perspiratory gland, blood flow, etc.).

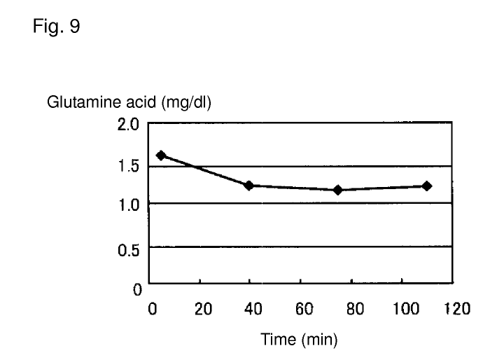

FIG. 9 is a view showing changes over time of the glutamic acid concentration in the perspiration after perspiration in the state shown in FIG. 8, assuming the second component is the glutamic acid. As mentioned above, the second component is a component in which the change in concentration is not related to the change in concentration of the first component in the blood. As shown in FIG. 8 and FIG. 9, the glutamic acid concentration demonstrates a constant value although the blood glucose value and the perspiration sugar value are rising after elapse of 40 minutes from the perspiration. Similar to the perspiration sugar value, the glutamic acid concentration is at high concentration compared to the concentration thereof after elapse of 40 minutes at the beginning of perspiration such as until elapse of 40 minutes from the perspiration in FIG. 6.

The fine behavior and the mechanism of the change in concentration of the component in the perspiration are not accurately known at the present stage. In particular, not only the sugar concentration, but the concentration of other components is also known to become higher at the beginning of perspiration than the concentration of after elapse of a predetermined time. Thus, when calculating the concentration of the component in the blood using the concentration of the component in the perspiration, the possibility that the obtained concentration of the component in the blood contains an error is high if the concentration of the component in the perspiration at the beginning of perspiration is used in the computation.

One factor that may cause the component in the perspiration to become high concentration at the beginning of perspiration is assumed to be because the transmissivity of the substance transmissive property of the perspiratory gland membrane temporarily increases at the beginning of perspiration, that is, the transmissive performance of the perspiratory gland membrane at the beginning of perspiration is higher than the transmissive performance after the beginning. With such explanation, various component concentrations in the perspiration become high, along with the first component and the second component, since the components greatly transmit through the perspiratory gland membrane at the beginning of perspiration compared with after the beginning of perspiration.

The measurement device 1 according to the present preferred embodiment uses such property and simultaneously measures the component (second component) other than the component (first component) to be measured in the measurement computation device 30. The change that depends on the state of the measurement site irrelevant to the blood concentration such as the influence of transmissive performance of the perspiratory gland membrane is corrected using the change in concentration of the second component in the conversion computation unit 309 to convert the concentration of the first component in the perspiration to the concentration of the first component in the blood.

The conversion method in the conversion computation unit 309 is not limited to a specific method, but includes the following example, as a specific example. In the conversion computation unit 309, the concentration of the first component in the blood may be obtained from the concentration of the first component and the concentration of the second component in the perspiration using the method other than the method described below.

The conversion computation unit 309 stores coefficients α, β, γ as coefficients defined in advance, corrects the concentration B1 of the sugar (glucose) serving as the first component with the concentration C1 of the glutamic acid serving as the second component in the perspiration input from the concentration calculation unit 307 using the following equation (1), and obtains the sugar concentration B in the perspiration after the correction:

B=B1−(αC1+β) Equation (1)

The sugar concentration B in the perspiration after the correction is converted to the sugar concentration A in the blood using the following equation (2):

A=γB Equation (2)

The coefficients α, β, γ may be obtained at the time of computation, etc., by the conversion computation unit 309 in place of those stored in advance. For instance, the coefficients may be determined by the conversion computation unit 309 from the perspiration sugar value, the glutamic acid concentration in the perspiration, and the concentration obtained by measuring the blood glucose value, many times over when the blood glucose value is relatively stable such as when the stomach is empty, and the like. Furthermore, only the coefficients α, β (for equation (1)) for correcting the perspiration sugar value from the measurement results of a great number of people may be determined for a great number of people, and the coefficient γ (for equation (2)) for converting the perspiration sugar value after the correction to the blood glucose value from the perspiration sugar value, the glutamic acid concentration in the perspiration, and the concentration obtained by measuring the blood glucose value one time when the stomach is empty for an individual.

When the conversion computation unit 309 converts the sugar concentration Bin the perspiration after the correction to the sugar concentration A in the blood using the following equations (3) and (4) in place of the equations (1) and (2), the coefficients α, γ in the equations (3) and (4) may be determined from the perspiration sugar value, the glutamic acid concentration in the perspiration, and the concentration obtained by measuring the blood glucose value, one time when the stomach is empty:

B=B1−αC1 Equation (3)

A=γB Equation (4)

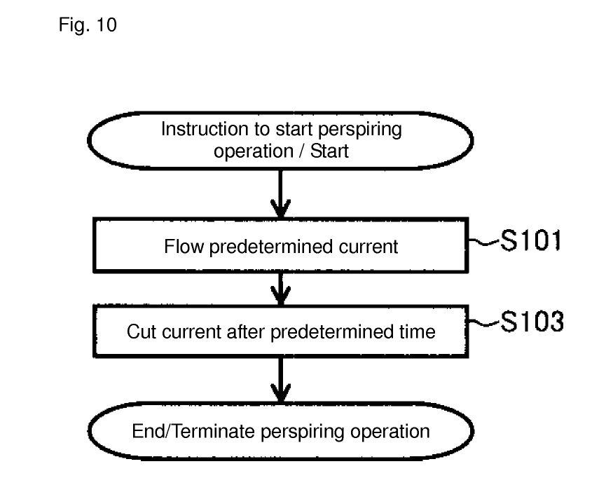

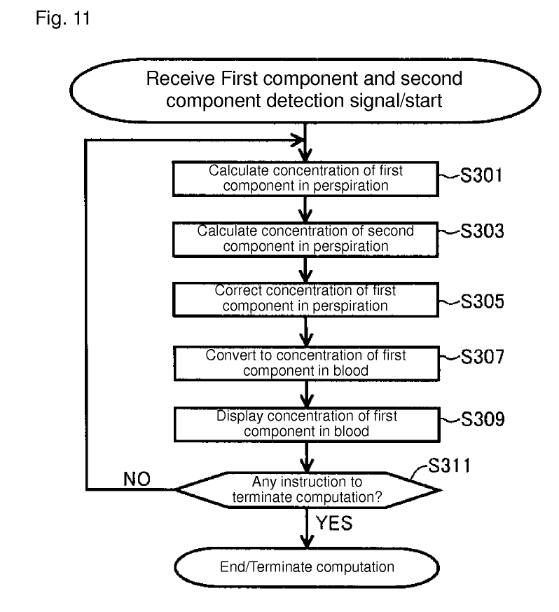

The flow of processes in the measurement device 1 will now be described. FIG. 10 is a flowchart showing the flow of the perspiring operation in the perspiration device 10. FIG. 11 is a flowchart showing the flow of the measurement computation operation in the measurement computation device 30. The processes shown in the flowcharts may be implemented by causing the control units 15, 35 to execute a predetermined computation program, and control each unit shown in FIGS. 2A, 2B, 3A, 3B to exhibit the functions shown in FIGS. 6, 7.

First, the perspiring operation shown in FIG. 10 starts when the sponge 41 with the solution containing the perspiration accelerator such as the pilocarpine solution is attached to the medical agent region 12A, the introducing electrode 11 is attached so as to contact the sponge 41, and then the operation to start the perspiring operation is carried out with the operation unit after attaching the perspiration device 10 to the measurement site using the belt 2A so that the sponge 41 contacts the skin 100. When the input of the operation signal from the operation unit is accepted by the operation input unit 101, the control unit 103 performs a process to generate the current for flowing a predetermined DC current from the introducing power 11 to the reference electrode 13 at the current generation unit 103, and flows a predetermined current between the electrodes (step S101). After elapse of a predetermined time from the start of the perspiring operation is detected or when accepting the input of the operation signal indicating the operation of operation termination at the operation input unit 101 after elapse of a predetermined time, the control unit 103 terminates the generation of the current at the current generation unit 105, and cuts the current flowing between the electrodes (step S103).

The perspiring operation in the perspiration device 10 is then terminated. Thereafter, the subject resolves the attachment state of the perspiration device 10 and detaches the sponge 41 from the skin 100, which is the measurement site, and cleans the skin 100. The subject then attaches the measurement computation device 30 at the same position using the belt 2B. The sponge 43 of the perspiration collection region 32 collects the perspiration perspired from the skin 100 to which the pilocarpine solution is infiltrated.

The measurement computation operation in the measurement computation device 30 may be started when the instruction to start the measurement computation operation is made at the operation unit with the measurement computation device 30 attached to the measurement site, or with the device attached for a constant time and detached after the perspiration is collected by the sponge 43, may be started when detected that the collected perspiration amount reached a predetermined amount by the liquid sensor 38 shown in FIG. 5, or may be started when the detection signal corresponding to the detection amount of the first component and the second component is input to the concentration calculation unit 307 from the first component detection unit 303 and the second component detection unit 305. The measurement computation operation in the measurement computation device 30 shown in FIG. 11 is started when the detection signal corresponding to the detection amount of the first component and the second component is input to the concentration calculation unit 307 from the first component detection unit 303 and the second component detection unit 305, and terminated when the operation signal for terminating the computation is input from the operation unit.

With reference to FIG. 11, when receiving the detection signal corresponding to the detection amount of the first component and the second component from the first component detection unit 303 and the second component detection unit 305, the concentration calculation unit 307 calculates the concentration of the first component and the concentration of the second component in the perspiration from such detection signals (steps S301, S303). The conversion computation unit 309 corrects the concentration of the first component in the perspiration calculated in step S301 using the concentration of the second component in the perspiration calculated in step S303 in equation (1), and obtains the concentration of the first component of after the correction (step S305). Furthermore, the concentration of the first component in the perspiration of after the correction obtained in step S305 is converted to the concentration of the first component in the blood in equation (2) (step S307), and input to the display unit 311. At the display unit 311, a process of displaying the computation result on the display 37 is executed, and the concentration of the first component in the blood obtained in step S307 is displayed (step S309).

The processes of steps S301 to S309 are repeated at a predetermined interval until the operation of terminating the conversion computation operation is made, and the concentration of the first component in the blood is displayed at a predetermined interval. Then, when the operation signal for terminating the conversion computation operation is input from the operation unit (YES in step S311), the conversion computation operation in the measurement computation device 30 is terminated.

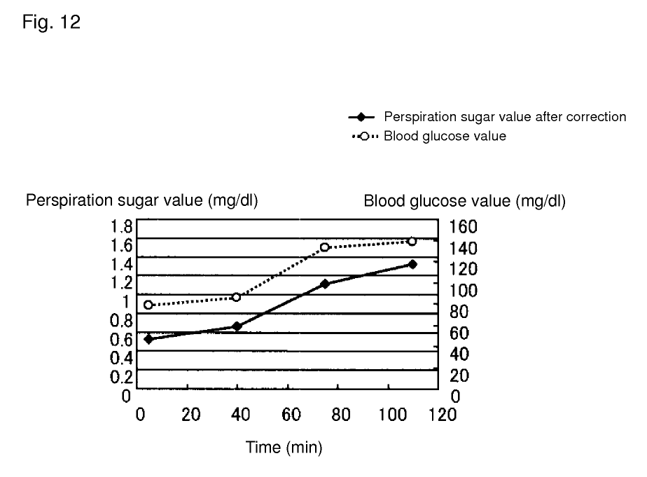

FIG. 12 is a view showing changes over time of the concentration in the perspiration of after correction and the blood glucose value corrected using the concentration in the perspiration of the glutamic acid as the second component by performing the process of step S305 with respect to the actual sugar concentration in the perspiration. It is apparent from FIG. 12 that the corrected perspiration sugar value demonstrates substantially the same behavior as the blood glucose value from the beginning of perspiration and thereafter by performing the correction. Therefore, compared with the relationship of the behavior between the actually measured (non-corrected) perspiration sugar value and the blood glucose value shown in FIG. 8, such behaviors are related at the beginning of the perspiration and the effects of the correction are exhibited.

That is, for the variation in change between the concentration in the perspiration and the concentration in the blood in that the concentration in the perspiration of the component (first component) to be measured is high particularly at the beginning of perspiration and that the concentration in the perspiration and the concentration in the blood do not demonstrate the relevance seen after the beginning of perspiration, the measurement device 1 according to the present preferred embodiment detects other components (second component) in addition to the first component and calculates the concentration thereof, and corrects the concentration of the first component using the concentration of the second component from the standpoint that the variation is made due to the transmissive performance of the perspiratory gland membrane. As a result, the influence of the transmissive performance of the perspiratory gland membrane can be removed from the concentration in the perspiration of the measured first component. Alternatively, the influence of the transmissive performance of the perspiratory gland membrane can be suppressed. In the measurement device 1, the concentration of the first component in the blood of high accuracy with few variations can be obtained by converting the concentration of the first component in the perspiration after the correction to the first component in the blood using the predetermined coefficient. The concentration of the first component in the blood of high accuracy with few variations can be obtained from the concentration of the first component in the perspiration by using the computation method performed in the measurement device 1.

Furthermore, the correction of high concentration at the beginning of perspiration has been mainly described in the above example, but the transmissive performance of the perspiratory gland membrane is assumed to change by the change in daily health conditions and skin state, where application can be made to the correction of the change in a long term standpoint of daily level.

In the example described above, one second component (glutamic acid in the specific example) is used as another component of the first component, but a plurality of perspiration components may be used for the second component. For instance, if the first component is sugar, at least one of the amino acids such as glutamic acid, lysine, glutamine, and asparagine acid, or at least one of such amino acids, calcium, and kalium may be used for the second component.

The preferred embodiments disclosed herein are illustrative in all aspects and should not be construed as being exclusive. The scope of the present invention is defined by the claims rather than by the description made above, and meanings equivalent to the claims and all modifications within the scope of the present invention are to be encompassed.

While preferred embodiments of the present invention have been described above, it is to be understood that variations and modifications will be apparent to those skilled in the art without departing the scope and spirit of the present invention. The scope of the present invention, therefore, is to be determined solely by the following claims.

What is claimed is:

1. A blood component concentration measurement device comprising:
a perspiration accelerating unit arranged to accelerate perspiration from a surface of a body or a measurement site;
a first measurement unit arranged to measure a concentration in the perspiration of a first component contained in the perspiration from the surface of the body or the measurement site;
a second measurement unit arranged to measure a concentration in the perspiration of a second component, different from the first component, contained in the perspiration from the measurement site;
a correction unit arranged to correct the concentration in the perspiration of the first component using the concentration in the perspiration of the second component; and
a conversion unit arranged to convert a result corrected by the correction unit to the concentration of the first component in a blood of the body.

2. The blood component concentration measurement device according to claim 1, wherein the second component is a component in which a rate reabsorbed by a perspiration tube is smaller than a predetermined value.

3. The blood component concentration measurement device according to claim 1, wherein the second component is a component in which relevance between a change in concentration in the perspiration and a change in concentration of the first component in the blood is lower than a predetermined correlation coefficient.

4. The blood component concentration measurement device according to claim 1, wherein the second component is a component in which a rate of a change in the concentration in the blood is smaller than a rate of a change in the concentration in the blood of the first component.

5. The blood component concentration measurement device according to claim 1, wherein the first component is sugar, and the second component is at least one of glutamine acid, lysine, glutamine, asparagine acid, calcium, and kalium.

6. A measurement method performed by a blood component concentration measurement device which includes an acquiring device arranged to acquire perspiration from a measurement site of a body, a detection device arranged to detect a component in the perspiration, and a computation device arranged to perform a computation using a value obtained from the component, the method comprising the steps of:
detecting a first component from the perspiration with the detection device;
calculating a concentration in the perspiration of the first component with the computation device;
detecting a second component, different from the first component, from the perspiration with the detection device;
calculating a concentration in the perspiration of the second component with the computation device;
correcting the concentration in the perspiration of the first component using the concentration in the perspiration of the second component with the computation device;
converting a result corrected in the correcting step to the concentration of the first component in the blood of the body with the computation device; and
executing a process of outputting the concentration in the blood of the first component with the computation device.
